(19) United States
(12) Patent Application Publication
(10) Pub. No.: US 2005/0074473 A1
(43) Pub. Date: Apr. 7, 2005

(54) Soft-focus cosmetic composition comprising fumed alumina

(75) Inventors: Laurent P. Kosbach, Colombes (FR); Elizabeth S. Sims, Champaign, IL (US)

Correspondence Address: Michelle B. Lando;Cabot Corporation, Billerica Technical Center, 157 Concord Road, Billerica, MA, 01821-7001, US

(73) Assignee: Cabot Corporation, Boston, MA (US)

(21) Appl. No.: 10/681,803

(22) Filed: Oct. 7, 2003

(51) Int. Cl. A61K 7/021; A61K 7/00

(52) U.S. Cl. 424/401; 424/63

(57) ABSTRACT

A soft-focus cosmetic composition comprising about 3 wt. % or more fumed alumina particles, a method of enhancing the soft-focus effect of a cosmetic composition comprising combining the cosmetic composition with about 3 wt. % or more fumed alumina particles, and a method for disguising skin imperfections, which method comprises applying a cosmetic composition to the skin, wherein the cosmetic composition comprises about 3 wt. % or more fumed alumina particles.

### FIELD OF THE INVENTION

This invention pertains to cosmetic compositions and methods for enhancing the soft-focus effect of cosmetic compositions and for disguising skin imperfections using soft-focus cosmetic compositions.

### BACKGROUND OF THE INVENTION

In an aging population, the demand for cosmetic products that hide the effects of aging has significantly increased. Wrinkles and fine lines, the primary signs of aging, cause the skin tone to look uneven by trapping light in the crevices formed by the wrinkles. The trapped light is absorbed and causes the appearance of dark spots. Unfortunately, many foundations and make-ups currently available on the market actually accentuate fine lines and wrinkles due to migration of the pigments into the wrinkle crevices. Other make-up compositions effectively cover up the imperfections, but create an unnatural, caked-on appearance.

A more recent approach to covering up the signs of aging involves the use of light-diffusing particles, which hide skin imperfections while projecting the natural skin tone. This approach has come to be known as the “soft-focus” effect. The criteria for a light-diffusing particle, which if met would provide the optimum diffusion or soft-focus effect, are outlined in Emmert, “Quantification of the Soft-Focus Effect,” Cosmetics and Toiletries, 111,57-61 (1996). The criteria are as follows: (1) the diffusive particle needs to have minimal light absorption, (2) the diffusive particle needs to have high total light transmission to provide a natural appearance, (3) most of the light transmission needs to be diffuse, so that the light reflected from the skin appears to be evenly distributed, (4) the specular reflection must be minimal so as to minimize luster that would increase the appearance of wrinkles, and (5) the scattered reflection component of the total reflection needs to be high in order to have an even light distribution over the area independent of underlying wrinkles (Emmert, supra, at p.58).

Cosmetic compositions have employed various light-diffusing particles for the purpose of achieving a soft-focus effect. Such particles include boron nitride particles, nylon particles, flake-like or plate-like alumina particles coated with a polymer or other materials, and spherical silica particles. However, there continues to be a need for additional methods and compositions for achieving a soft-focus effect in cosmetics. The present invention provides such a method and composition. These and other advantages of the invention, as well as additional inventive features, will be apparent from the description of the invention provided herein.

### BRIEF SUMMARY OF THE INVENTION

The invention provides a soft-focus cosmetic composition comprising about 3 wt. % or more fumed alumina particles. The invention also provides a method of enhancing the soft-focus effect of a cosmetic composition comprising combining the cosmetic composition with about 3 wt. % or more fumed alumina particles. A method for disguising skin imperfections also is provided herein, which method comprises applying a cosmetic composition to the skin, wherein the cosmetic composition comprises about 3 wt. % or more fumed alumina particles.

### BRIEF DESCRIPTION OF THE DRAWINGS

FIG. 1 is a graph of the percent transmission of light through a control sample of polymer without any light-diffusing particles at wavelengths of light ranging from 400-700 nm. The square (▪) data points represent total transmission measurements, and the triangular data points (▴) represent specular transmission measurements.

FIG. 2 is a graph of the percent reflection of light from a control sample of polymer without any light-diffusing particles at wavelengths of light ranging from 400-700 nm. The diamond (♦) data points represent total reflectance measurements, and the square data points (▪) represent scattered reflectance measurements.

FIG. 3 is a graph of the percent transmission of light through a sample of polymer containing titanium dioxide particles at wavelengths of light ranging from 400-700 nm. The square (▪) data points represent total transmission measurements, and the triangular data points (▴) represent specular transmission measurements.

FIG. 4 is a graph of the percent reflection of light from a control sample of polymer containing titanium dioxide particles at wavelengths of light ranging from 400-700 nm. The diamond (♦) data points represent total reflectance measurements, and the square data points (▪) represent scattered reflectance measurements.

FIG. 5 is a graph of the percent transmission of light through a sample of polymer containing boron nitride particles at wavelengths of light ranging from 400-700 nm. The square (▪) data points represent total transmission measurements, and the triangular data points (▴) represent specular transmission measurements.

FIG. 6 is a graph of the percent reflection of light from a control sample of polymer containing boron nitride particles at wavelengths of light ranging from 400-700 nm. The diamond (♦) data points represent total reflectance measurements, and the square data points (▪) represent scattered reflectance measurements.

FIG. 7 is a graph of the percent transmission of light through a sample of polymer containing nylon 12 particles at wavelengths of light ranging from 400-700 nm. The square (▪) data points represent total transmission measurements, and the triangular data points (▴) represent specular transmission measurements.

FIG. 8 is a graph of the percent reflection of light from a control sample of polymer containing nylon 12 particles at wavelengths of light ranging from 400-700 nm. The diamond (♦) data points represent total reflectance measurements, and the square data points (▪) represent scattered reflectance measurements.

FIG. 9 is a graph of the percent transmission of light through a sample of polymer containing fumed alumina particles at wavelengths of light ranging from 400-700 nm. The square (▪) data points represent total transmission measurements, and the triangular data points (▴) represent specular transmission measurements.

FIG. 10 is a graph of the percent reflection of light from a control sample of polymer containing fumed alumina particles at wavelengths of light ranging from 400-700 nm. The diamond (♦) data points represent total reflectance measurements, and the square data points (▪) represent scattered reflectance measurements.

### DETAILED DESCRIPTION OF THE INVENTION

The invention provides a soft-focus cosmetic composition comprising about 3 wt. % or more fumed alumina particles. The term “fumed alumina” is used herein to refer to a form of alumina that is comprised of substantially spherical primary particles that are fused or aggregated into larger, irregularly shaped aggregate particles. The aggregate bonds between the smaller primary particles are strong; thus, substantial force is required to break the aggregate particles into smaller particles. As a result, the aggregate particles do not typically break down when the particles are dispersed in a carrier. The aggregate particles are usually associated with one another to form even larger agglomerate particles, which are held together by weaker bonds (e.g., hydrogen bonding). Agglomerated particles, thus, can break down into aggregate particles upon dispersion depending upon the amount of dispersion energy used. The fumed alumina particles are substantially different in form, appearance, and physical and/or chemical characteristics from other forms of alumina, such as plate-like or flake-like alumina. While these forms of alumina may form loosely associated agglomerate particles, they are not comprised of aggregate particles, nor are the primary particles spherical in shape.

As used herein, the term “fumed alumina” encompasses pyrogenic alumina. Fumed alumina can be produced by known methods, such as by fuming processes and other pyrogenic and flame-type processes known in the art, and is commercially available (e.g., SpectraAl™, manufactured by Cabot Corporation). Fumed alumina used in conjunction with the soft-focus cosmetic composition of the invention typically has a BET surface area of about 35 m²/g or more, such as about 40 m²/g or more, preferably about 45 m²/g or more (e.g., about 50 m²/g or more). The maximum surface area of fumed alumina used in conjunction with the soft-focus cosmetic composition of the invention typically is about 200 m²/g or less, and preferably about 150 m²/g or less, such as about 100 m²/g or less (e.g., about 75 m²/g or less), or even about 65 m²/g or less (e.g., about 60 m²/g or less).

The primary particles of fumed alumina typically comprise crystalline and amorphous alumina. The degree of crystallinity, as well as the relative amount of the various different types of crystalline phases of alumina present, will depend, at least in part, upon the method by which the fumed alumina is produced. Preferred fumed alumina particles comprise a combined δ*-phase and θ-phase crystalline alumina content of about 30% or more, more preferably about 40% or more (e.g., about 45% or more), or even about 55% or more (e.g., about 60% or more). The crystalline content of fumed alumina can be determined by any suitable method known in the art, such as by X-ray diffraction.

The soft-focus cosmetic composition of the invention comprises about 3 wt. % or more fumed alumina particles. While this amount of fumed alumina particles is believed to be sufficient to produce some degree of soft-focus effect in a cosmetic composition, preferred formulations according to the invention comprise more than 3 wt. % fumed alumina particles, such as about 5 wt. % or more fumed alumina particles (e.g., about 10 wt. % or more fumed alumina particles), or about 15 wt. % or more fumed alumina particles (e.g., about 20 wt. % or more fumed alumina particles), or even about 30 wt. % or more fumed alumina particles. Of course, the soft-focus composition of the invention can comprise other known cosmetically acceptable additives, as discussed below. Thus, in most formulations, the soft-focus cosmetic composition of the present invention will typically comprise about 95 wt. % or less fumed alumina particles (e.g., about 3-95 wt. % fumed alumina particles), such as about 85 wt. % or less fumed alumina particles (e.g., about 5-85 wt. % fumed alumina particles), or about 75 wt. % or less fumed alumina particles (e.g., about 15-75 wt. % fumed alumina particles). It is to be understood that all percent compositions referred to herein pertain to the percent by weight of the dry cosmetic composition unless specifically stated otherwise. Thus, in the case of wet formulations (e.g., liquids, emulsions, lotions, creams, etc.), the percent compositions referred to herein pertain to the weight percent of the composition after drying by a suitable method.

The fumed alumina particles used in conjunction with the invention can have any suitable particle size. Particle size, as used herein, refers to the spherical diameter of the particle, which is the diameter of the smallest sphere that could encompass the particle.

Typically, fumed alumina particles that are useful in conjunction with the invention will have an average aggregate particle size (by number) of about 50 nm or more, such as about 75 nm or more (e.g., about 100 nm or more), or even about 125 nm or more (e.g., 140 nm or more). The fumed alumina particles typically have an average aggregate particle size (by number) of about 300 nm or less, for example, about 200 nm or less, or even about 175 nm or less (e.g., about 160 nm or less). For some applications, it may be preferable to use fumed alumina particles that have a narrow aggregate particle size distribution. For instance, it is sometimes preferred to use fumed alumina particles with a particle size distribution such that about 70% or more, or even about 80% or more (e.g., about 95 wt. % or more) of the fumed alumina particles have an aggregate particle size of 300 nm or less, such as 200 nm or less (e.g., 175 nm or less). Similarly, it is sometimes preferred to use a particle size distribution wherein about 70% or more, or even about 80% or more (e.g., about 95 wt. % or more) of the fumed alumina particles have an aggregate particle size of about 50 nm or more, such as about 75 nm or more (e.g., about 100 nm or more), or even about 125 nm or more (e.g., 140 nm or more).

The fumed alumina aggregate particles can be present in the cosmetic composition of the invention as larger, agglomerated particles. The cosmetic composition of the invention preferably comprises agglomerate particles of fumed alumina having an average agglomerate particle size of about 5 μm or more, such as about 10 μm or more, or even about 15 μm or more. Particles that are too large can produce an undesirable texture in the cosmetic composition. Thus, it is preferable that the agglomerate fumed alumina particles have a maximum average particle size of about 30 μm or less, such as about 25 μm or less, or even about 20 μm or less. For some applications, it may be preferable to use fumed alumina particles that have a narrow agglomerate particle size distribution. For instance, the cosmetic composition of the invention can comprise fumed alumina particles with a particle size distribution such that about 70% or more, or even about 80% or more (e.g., about 95 wt. % or more) of the fumed alumina particles have an agglomerate particle size of 5 μm or more, such as 10 μm or more, or even 15 μm or more. Similarly, the cosmetic composition of the invention can comprise fumed alumina particles with a particle size distribution such that about 70% or more, or even about 80% or more (e.g., about 95 wt. % or more) of the fumed alumina particles have an agglomerate particle size of 30 μm or less, such as 25 μm or less, or even 20 μm or less. Of course, the cosmetic composition of the invention can comprise a mixture of aggregate and agglomerate particles.

The fumed alumina particles used in conjunction with the invention can be treated or untreated. Possible treatments include hydrophobicizing treatments (e.g., hydrophobic fumed alumina), as well as treatments to alter the surface charge characteristics (e.g., cationic or anionic treatments).

The invention contemplates the use of other light diffusing particles in combination with the fumed alumina particles. Useful light diffusing particles include boron nitride particles, nylon particles (e.g., nylon 12 particles), fumed silica particles, spherical silica particles, and other various light-diffusing agents.

The soft-focus cosmetic composition of the invention can be formulated as any type of skin treatment or makeup product. Skin treatment product formulations of the invention include lip products, acne treatments, moisturizers, anti-aging products, lifting treatments, cellulite treatments, and eye treatments. Makeup product formulations of the invention include, but are not limited to, foundations, blushes, pressed or loose powders, concealers, bronzers, eyeshadows, eyeliners, lipsticks, and lip glosses. The products of the invention can take any form which is typical of cosmetic products, for example, hot pour formulations, water-in-oil emulsions, oil-in-water emulsions, gels, sticks, sprays, anhydrous formulations, and pressed or loose powders. There is no limitation on the type of vehicle that can be employed. In particular, the preferred identity of the vehicle will be largely controlled by the type of product into which the components are to be incorporated. For a liquid foundation, for example, a water-in-oil emulsion is preferred for aesthetic reasons, and although the oil portion of the vehicle can be any which is typically used for this purpose, such as, for example, a volatile or non-volatile silicone oil. Preferably, the soft-focus cosmetic composition of the invention is a powder foundation.

The soft-focus cosmetic composition of the invention also can comprise any other cosmetically and/or dermatologically acceptable ingredients. Pigments, such as interference pigments, inorganic pigments, and organic pigments can be used to adjust the color shade of the composition. Useful interference pigments are available commercially from a wide variety of suppliers. Examples of preferable interference pigments are Flonac MS-30C, which is mica treated with titania and iron oxide (yellow), and MU-10C, which is mica treated with titania (white). Interference pigments of different colors or types can be combined to blend an appropriate shade or intensity of color to match the natural skin tone. Examples of useful inorganic pigments include iron oxides (yellow, red, umber, brown, or black), ferric ammonium ferrocyanide (blue), manganese violet, ultramarine blue, chrome oxide (green), talc, lecithin modified talc, zeolite, kaolin, lecithin modified kaolin, titanium dioxide (white), zinc oxide, and mixtures thereof. In addition to providing color to match the color of the skin, titanium dioxide, zinc oxide, and iron oxide function as particulate inorganic sunscreens. Organic pigments can include natural colorants and synthetic monomeric and polymeric colorants. Exemplary are phthalocyanine blue and green pigments, diarylide yellow and orange pigments, and azo-type red and yellow pigments such as toluidine red, litho red, naphthol, red and brown pigments. Also useful are lakes, which are pigments, formed by the precipitation and absorption of organic dyes on an insoluble base, such as alumina, barium, or calcium hydrates. Water soluble colorants (such as FD&C Blue #1), oil soluble colorants (such as D&C Green #6), and stains (such as bromo dyes and fluorescein dyes) also can be employed. The amount and type of any non-diffusing pigment used will vary depending upon the nature of the final product and the desired intensity of color; generally, however, the amount of non-diffusing pigment will be about 1-10 wt. %, and preferably about 1-5 wt. %, by weight of the total composition. In addition, microfine particulate pigments can be used at somewhat higher levels than those of normal particle size without significantly increasing the level of opacity of the composition.

The formulation also can comprise other components that may be chosen depending on the carrier and/or the intended use of the formulation. Additional components include, but are not limited to, water soluble sunscreens (such as Eusolex 232), oil soluble sunscreens (such as octyl methoxycinnamate), and organic sunscreens (such as camphor derivatives, cinnamates, salicylates, benzophenones, triazines, PABA derivatives, diphenylacrylate derivatives, and dibenzoylmethane derivatives), antioxidants (such as BHT), chelating agents (such as disodium EDTA), emulsion stabilizers (such as carbomer), preservatives (such as methyl paraben), fragrances (such as pinene), flavoring agents (such as sorbitol), humectants (such as glycerine), waterproofing agents (such as PVP/Eicosene copolymer), water soluble film-formers (such as hydroxypropyl methylcellulose), oil-soluble film formers (such as hydrogenated C-9 Resin), moisturizing agents such as cholesterol), cationic polymers (such as Polyquatemium 10), anionic polymers (such as xanthan gum), pigment wetting agents (such as Arlacel™ P100 or Emerest™ 2452), vitamins (such as tocopherol), and the like.

The compositions also can comprise one or more active components. Examples of useful active components include, but are not limited to, those that improve or eradicate age spots, keratoses, and wrinkles, analgesics, anesthetics, anti-acne agents, antibacterials, antiyeast agents, antifungal agents, antiviral agents, antidandruff agents, antidermatitis agents, antipruritic agents, antiemetics, antimotion sickness agents, anti-inflammatory agents, antihyperkeratolytic agents, anti-dry skin agents, antiperspirants, antipsoriatic agents, antiseborrheic agents, hair conditioners and hair treatment agents, antiaging agents, antiwrinkle agents, antiasthmatic agents and bronchodilators, sunscreen agents, antihistamine agents, skin lightening agents, depigmenting agents, wound-healing agents, vitamins, corticosteroids, tanning agents, sunscreens, and hormones. More specific examples of useful active components include retinoids such as retinol, and esters, acids, and aldehydes thereof; ascorbic acid, and esters and metal salts thereof; tocopherol and esters and amide derivatives thereof; shark cartilage; milk proteins; alpha- or beta-hydroxy acids; DHEA and derivatives thereof; topical cardiovascular agents; and other various active ingredients including clotrimazole, ketoconazole, miconozole, griseofulvin, hydroxyzine, diphenhydramine, pramoxine, lidocaine, procaine, mepivacaine, monobenzone, erythromycin, tetracycline, clindamycin, meclocyline, hydroquinone, minocycline, naproxen, ibuprofen, theophylline, cromolyn, albuterol, hydrocortisone, hydrocortisone 21-acetate, hydrocortisone 17-valerate, hydrocortisone 17-butyrate, betamethasone valerate, betamethasone diproprionate, triaminolone acetonide, fluocinonide, clobetasol, proprionate, benzoyl peroxide, crotamiton, propranolol, and promethazine. The soft-focus cosmetic composition of the invention also can comprise a mixture of two or more of any of the above components.

The invention also provides a method of enhancing the soft-focus effect of a cosmetic composition (i.e., a method of preparing the soft-focus cosmetic composition of the invention) comprising combining a cosmetic composition with about 3 wt. % or more fumed alumina particles. There is no limitation on the type of cosmetic composition that can be used in accordance with this aspect of the invention. However, it is preferred that the cosmetic composition used is translucent as applied. The cosmetic composition can be combined with the fumed alumina particles in accordance with the invention by any method suitable for dispersing the fumed alumina particles in the cosmetic composition, such as by mixing or stirring. The fumed alumina particles and other components of the soft-focus cosmetic composition are as previously discussed. The discussion with respect to the features of the soft-focus cosmetic composition of the invention is applicable to the method of enhancing the soft-focus effect of a cosmetic composition.

As mentioned, the soft-focus cosmetic composition of the invention is useful for reducing or eliminating imperfections of the skin, such as wrinkles, blemishes, pores, scars, and the like. In this regard, the invention provides a method of disguising skin imperfections comprising applying a cosmetic composition to the skin, wherein the cosmetic composition comprises about 3 wt. % or more fumed alumina particles (i.e., a method of using the soft-focus cosmetic composition of the invention by applying it to the skin). The application of the cosmetic composition comprising about 3 wt. % or more fumed alumina particles to the skin causes the visible effect of the skin imperfections to be reduced or eliminated by scattering the light reflected from the skin to the observer. The fumed alumina particles used in conjunction with this method, as well as the other components of the soft-focus cosmetic composition, are as previously discussed. The discussion with respect to the features of the soft-focus cosmetic composition of the invention is applicable to the method of disguising skin imperfections.

### EXAMPLE

This example further illustrates the invention but, of course, should not be construed as in any way limiting its scope. In particular, this example demonstrates the effectiveness of fumed alumina as a light-diffusing, soft-focus additive in cosmetic formulations, and compares the performance of fumed alumina to other light-diffusing particles.

Four types of particles were tested for light-diffusing properties: titanium dioxide (Chroma-Philic™ marketed by Shield Manufacturing Compay), boron nitride (Tres BN™ PUHP1109 marketed by Saint Gobain Corporation), nylon 12 (Orgosol™ 2002 UP-NAT-COS marketed by Lipo Chemicals, Inc.), and fumed alumina (SpectraAl™ manufactured by Cabot Corporation). In order to evaluate the light-diffusing properties of the particles, each type of particle was incorporated into a separate thin polymer film. The films were made by dispersing 3 wt. % of the particles in 32 wt. % isopropanol, 32 wt. % de-ionized water, and 33 wt. % Polyderm™ PE-PA polyurethane (30% aqueous) (Alzo Inc.) with light agitation. The particle dispersions were then applied to glass slides using a 50 μm bird applicator. The resulting air-dried films were estimated to be 5 μm thick with the light-diffusing particles constituting approximately 23 wt. % of the dry film. The refractive index of the polymer (1.54) falls within the range of common cosmetic vehicles (1.33-1.6). A control was prepared using the same isopropanol/polymer composition without any light-diffusing particles. Table 1 provides a list of the samples tested, along with the refractive index and particle size of the particles included in each sample.

TABLE 1

| | | Refractive | |
|---|---|---|---|
| Sample | Particle Type | Index | Particle Size Range |
| A (control) | No particle | N/A | N/A |
| B (comparative) | Titanium | 2.50 | 5-14 μm |
| | Dioxide | | |
| C (comparative) | Boron Nitride | 1.74 | 6-15 μm |
| D (comparative) | Nylon 12 | 1.53 | 5-15 μm |
| E (invention) | Fumed Alumina | 1.71 | 10-30 μm* |

*Agglomerate particle size range; average aggregate particle size of 150 nm.

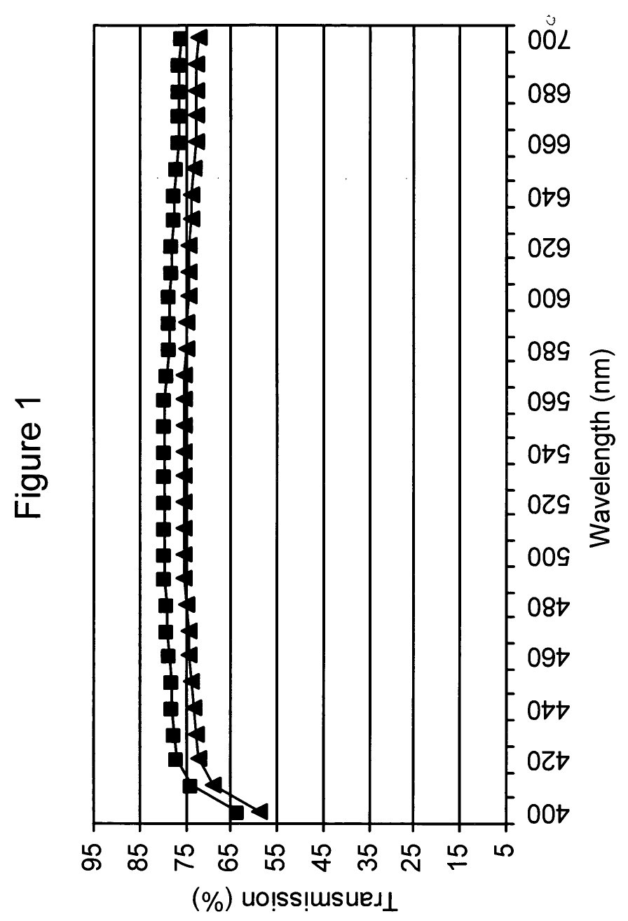

The optical properties of each sample were measured using a HunterLab UltraScan® XE. The UltraScan® XE is a dual beam xenon flash spectrophotometer with a wavelength range from 360 to 750 nm. The sensor uses an integrating sphere to measure reflected or transmitted light. The sphere contains a specular exclusion port allowing measurements that exclude the specular components. The following four types of measurements were taken for each type of particle evaluated: total transmission, specular transmission, total reflectance, and scattered reflectance. The measurements were taken over a range of wavelengths from 400-700 nm, as illustrated by FIGS. 1-10. An average value for each category was calculated. The results are provided in Table 2.

TABLE 2

| | Ave. Total | Ave. | | Ave. |
|---|---|---|---|---|
| | Trans- | Specular | Ave. Total | Scattered |
| | mission | Transmission | Reflectance | Reflectance |
| Sample | (%) | (%) | (%) | (%) |
| A (control) | 78 | 73 | 12 | 5 |
| B (comparative) | 61 | 26 | 30 | 25 |
| C (comparative) | 74 | 44 | 15 | 12 |
| D (comparative) | 84 | 33 | 12 | 10 |
| E (invention) | 83 | 38 | 12 | 10 |

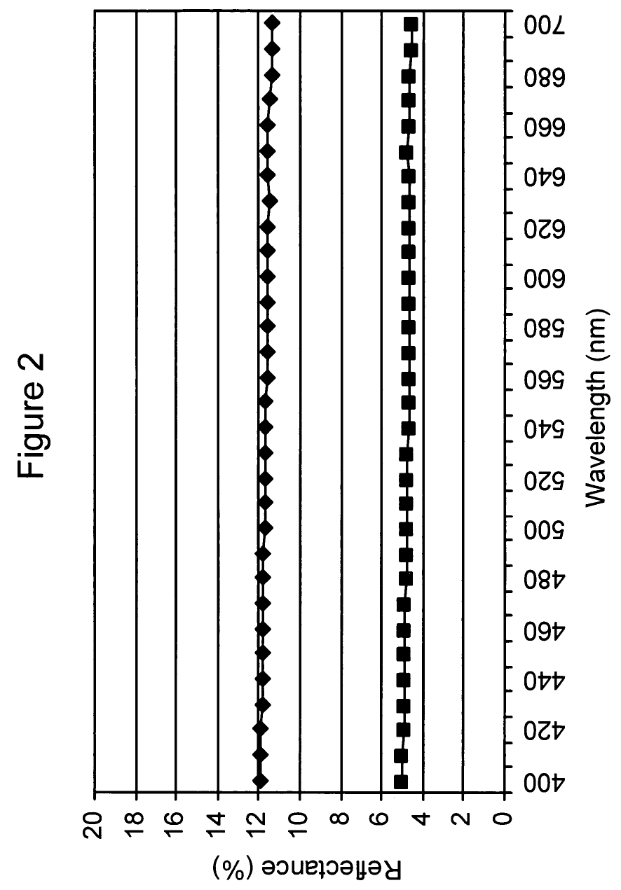

Control sample A (formulation with no polymer) had an average total transmission of 78%, with a specular (i.e., direct) transmission component equal to 94% of the average total transmission (FIG. 1). The high percentage of specular transmission is indicative of a film that is essentially transparent. The reflectance measurements for the control show a low percentage of scattered reflectance relative to the total reflectance (42% of the average total reflectance), which signifies a glossy film (FIG. 2).

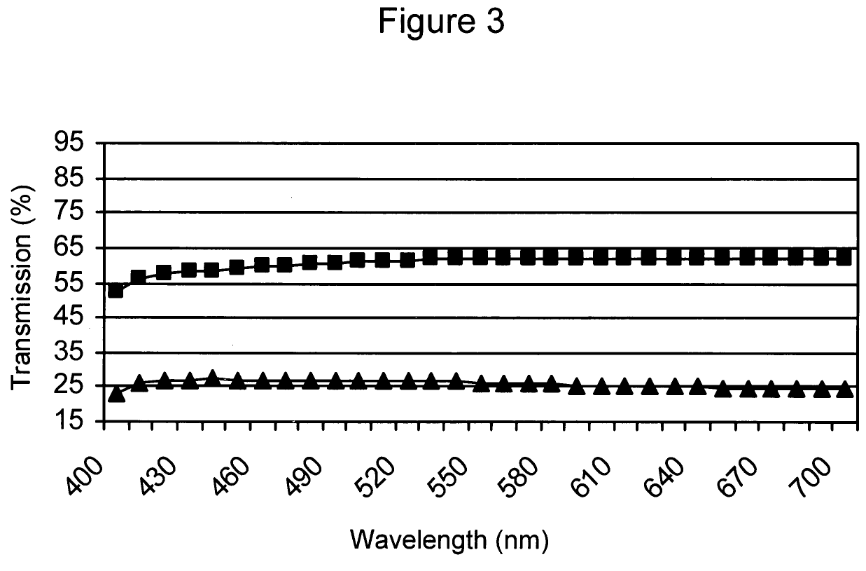

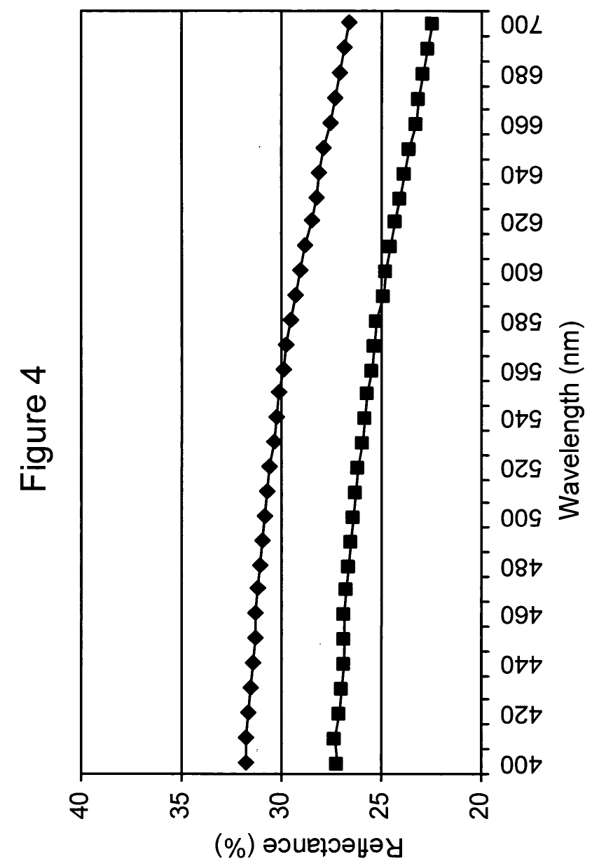

The transmission measurements of comparative sample B (titanium dioxide) (FIG. 3) demonstrate a total average transmission of only 61% with a relatively low specular transmission (42% of the average total transmission). This indicates good light diffusion (% diffusion=% total transmission−% specular transmission), but low overall transmission resulting in a high level of opacity. Sample B also exhibited high overall reflectance, 83% of which is present as scattered reflectance (FIG. 4). The high overall reflectance in conjunction with a high percentage of scattered reflection produces a white film appearance that is characteristic of compositions comprising titanium dioxide. These results indicate that titanium dioxide is not well suited for use as a soft-focus particle.

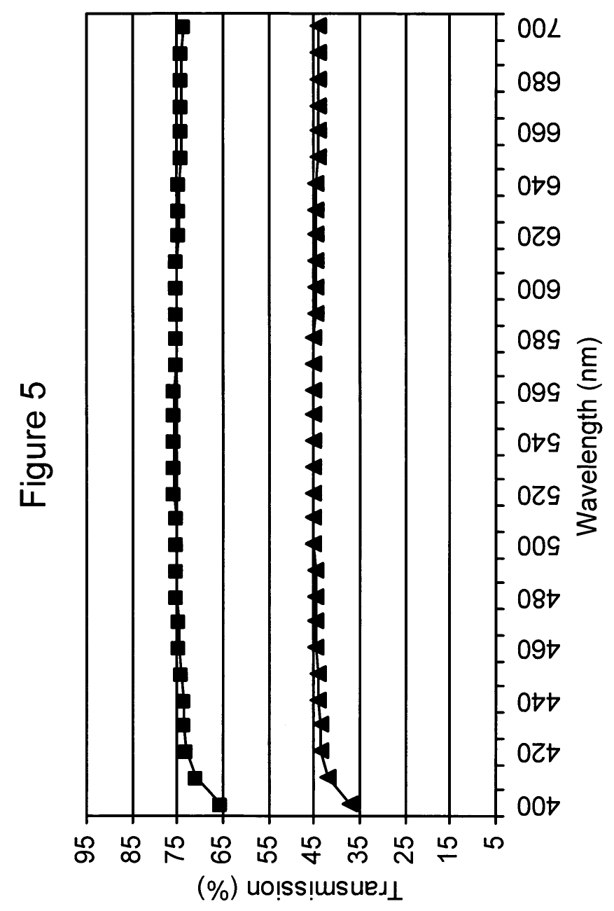

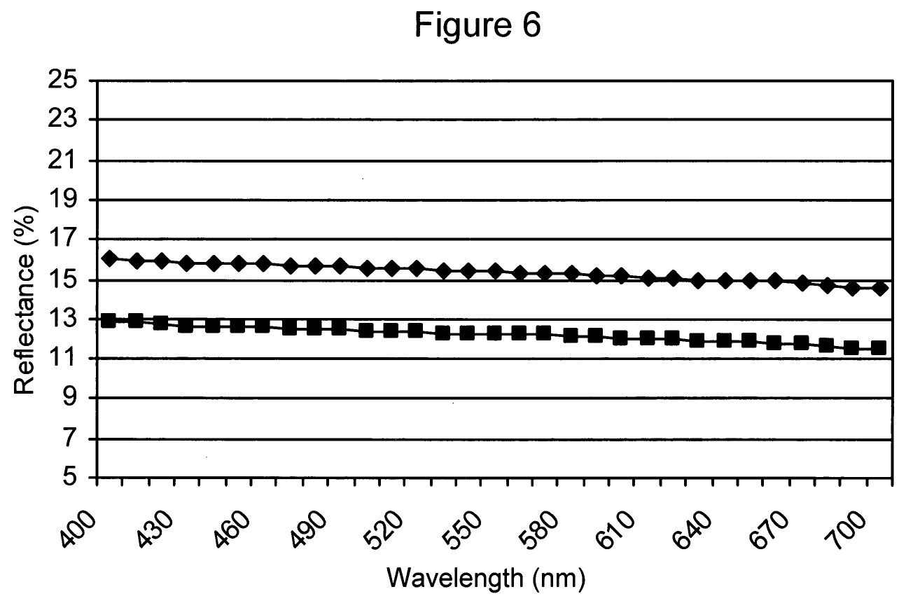

Comparative sample C (boron nitride) demonstrated significantly higher transparency (74% average total transmission) than sample B, making the film less opaque, but at the expense of higher specular transmission (59% of the average total transmission) and consequent loss of diffusion (FIG. 5). Also, the total reflectance is much lower for sample C, with a large portion of the total reflectance being scattered (FIG. 6). This indicates that boron nitride can be used to make a film with much lower degree of whiteness, minimizing the caked-on appearance, but with less light-diffusing ability. Accordingly, its usefulness as a soft-focus particle is somewhat limited.

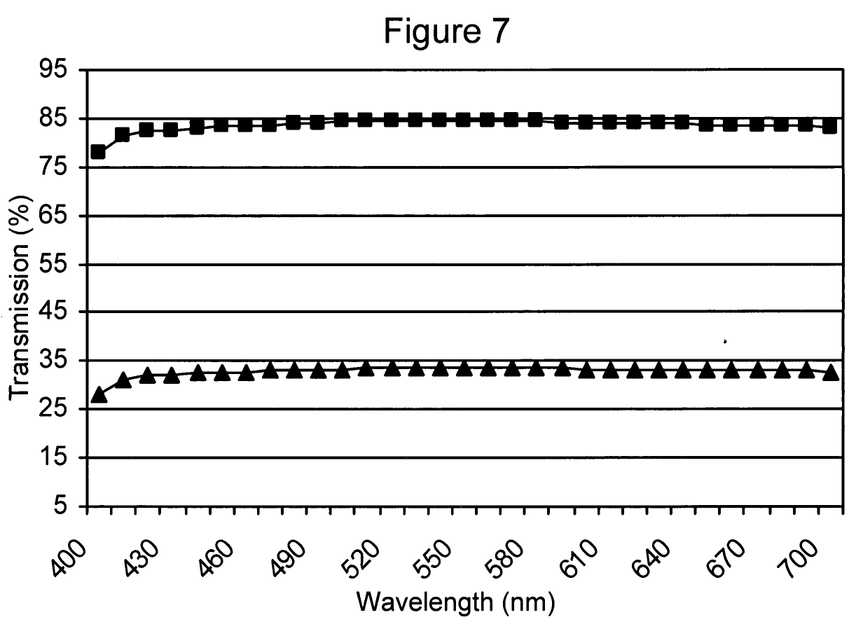

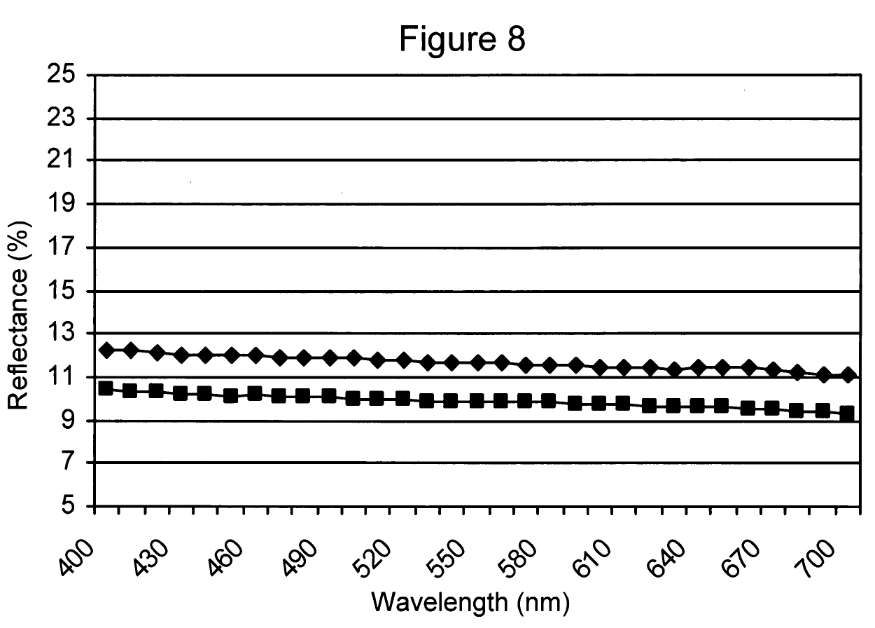

Comparative sample D (nylon 12) exhibited a high level of total transmission (84%) with a low percentage of specular transmission (40% of the average total transmission) (FIG. 7). Accordingly, sample D demonstrated a high degree of diffusion. Sample D also exhibited a total reflectance of <15% with a high contribution from the scattered reflection component (83% of the average total reflectance) (FIG. 8). The optical characteristics of nylon 12 demonstrate its ability to provide a film that minimizes a white, opaque appearance, while retaining good light-diffusing properties.

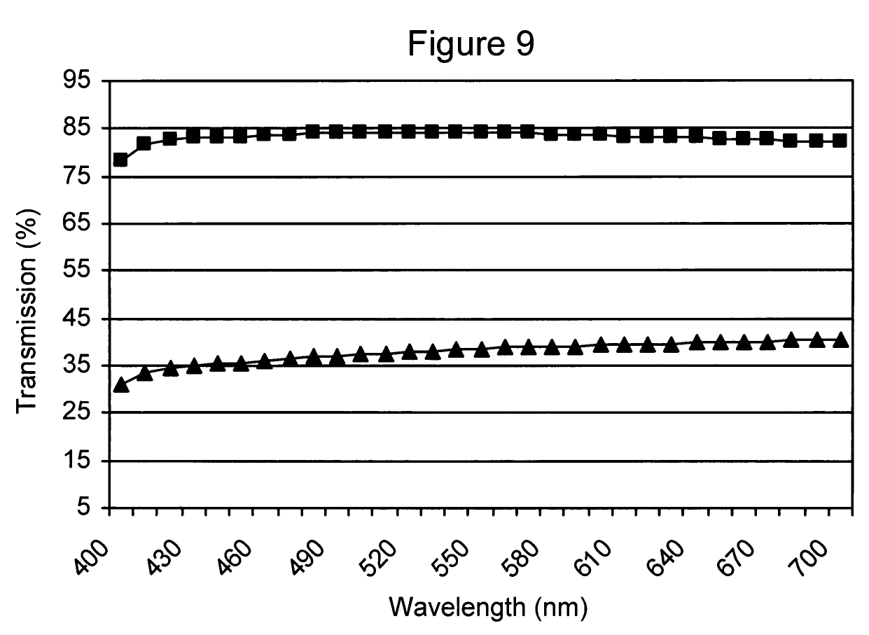

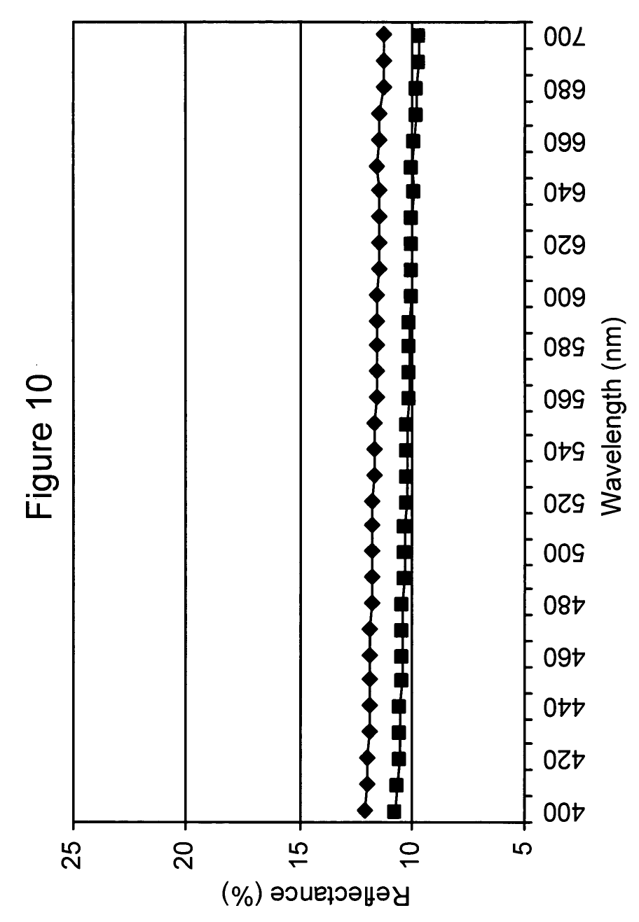

Invention sample E (fumed alumina) also demonstrated a very high total transmission (85%), a small specular transmission component (46% of the total transmission), and low overall reflectance with a high percentage of the total (83% of the total reflectance) being scattered reflection (FIGS. 9 and 10). Thus, fumed alumina meets or exceeds all the behaviors required to provide excellent soft-focus effects.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A soft-focus cosmetic composition comprising about 3 wt. % or more fumed alumina particles.

2. The soft-focus cosmetic composition of claim 1, wherein the cosmetic composition comprises about 5 wt. % or more fumed alumina particles.

3. The soft-focus cosmetic composition of claim 2, wherein the cosmetic composition comprises about 15 wt. % or more fumed alumina particles.

4. The soft-focus cosmetic composition of claim 3, wherein the cosmetic composition comprises about 30 wt. % or more fumed alumina particles.

5. The soft-focus cosmetic composition of claim 1, wherein the fumed alumina particles have an average aggregate particle size (by number) of about 50 nm or more.

6. The soft-focus cosmetic composition of claim 5, wherein the fumed alumina particles have an average aggregate particle size (by number) of about 300 nm or less.

7. The soft-focus cosmetic composition of claim 6, wherein about 70 wt. % or more of the fumed alumina particles have an aggregate particle size of 300 nm or less.

8. The soft-focus cosmetic composition of claim 1, wherein the fumed alumina particles have an average agglomerate particle size (by number) of about 5 μm or more.

9. The soft-focus cosmetic composition of claim 8, wherein the fumed alumina particles have an average agglomerate particle size (by number) of about 30 μm or less.

10. The soft-focus cosmetic composition of claim 8, wherein about 70 wt. % or more of the fumed alumina particles have an agglomerate particle size of 5 μm or more.

11. The soft-focus cosmetic composition of claim 9, wherein about 70 wt. % or more of the fumed alumina particles have an agglomerate particle size of 30 μm or less.

12. The soft-focus cosmetic composition of claim 1, wherein the fumed alumina particles comprise a combined δ*-phase and θ-phase crystalline alumina content of about 30% or more.

13. A method of enhancing the soft-focus effect of a cosmetic composition comprising combining the cosmetic composition with about 3 wt. % or more fumed alumina particles.

14. The method of claim 13, wherein the cosmetic composition comprises about 5 wt. % or more fumed alumina particles.

15. The method of claim 14, wherein the cosmetic composition comprises about 15 wt. % or more fumed alumina particles.

16. The method of claim 15, wherein the cosmetic composition comprises about 30 wt. % or more fumed alumina particles.

17. The method of claim 13, wherein the fumed alumina particles have an average aggregate particle size (by number) of about 50 nm or more.

18. The method of claim 17, wherein the fumed alumina particles have an average aggregate particle size (by number) of about 300 nm or less.

19. The method of claim 18, wherein about 70 wt. % or more of the fumed alumina particles have an aggregate particle size of 300 nm or less.

20. The method of claim 13, wherein the fumed alumina particles have an average agglomerate particle size (by number) of about 5 μm or more.

21. The method of claim 20, wherein the fumed alumina particles have an average agglomerate particle size (by number) of about 30 μm or less.

22. The method of claim 20, wherein about 70 wt. % or more of the fumed alumina particles have an agglomerate particle size of 5 μm or more.

23. The method of claim 21, wherein about 70 wt. % or more of the fumed alumina particles have an agglomerate particle size of 30 μm or less.

24. The method of claim 13, wherein the fumed alumina particles comprise a combined δ*-phase and θ-phase crystalline alumina content of about 30% or more.

25. A method for disguising skin imperfections comprising applying a cosmetic composition to the skin, wherein the cosmetic composition comprises about 3 wt. % or more fumed alumina particles.

26. The method of claim 25, wherein the cosmetic composition comprises about 5 wt. % or more fumed alumina particles.

27. The method of claim 26, wherein the cosmetic composition comprises about 15 wt. % or more fumed alumina particles.

28. The method of claim 27, wherein the cosmetic composition comprises about 30 wt. % or more fumed alumina particles.

29. The method of claim 25, wherein the fumed alumina particles have an average aggregate particle size (by number) of about 50 nm or more.

30. The method of claim 29, wherein the fumed alumina particles have an average aggregate particle size (by number) of about 300 nm or less.

31. The method of claim 30, wherein about 70 wt. % or more of the fumed alumina particles have an aggregate particle size of 300 nm or less.

32. The method of claim 25, wherein the fumed alumina particles have an average agglomerate particle size (by number) of about 5 μm or more.

33. The method of claim 32, wherein the fumed alumina particles have an average agglomerate particle size (by number) of about 30 μm or less.

34. The method of claim 32, wherein about 70 wt. % or more of the fumed alumina particles have an agglomerate particle size of 5 μm or more.

35. The method of claim 33, wherein about 70 wt. % or more of the fumed alumina particles have an agglomerate particle size of 30 μm or less.

36. The method of claim 25, wherein the fumed alumina particles comprise a combined δ*-phase and θ-phase crystalline alumina content of about 30% or more.
